(19) United States
(12) Patent Application Publication
(10) Pub. No.: US 2026/0045350 A1
(43) Pub. Date: Feb. 12, 2026

(54) SYSTEM FOR PROVIDING DIGITAL THERAPEUTIC AGENT TO TREAT TEMPOROMANDIBULAR JOINT DISEASE OF PATIENT

(71) Applicant: Beyondmedicine Co.,Ltd., Seoul (KR)

(72) Inventor: Dae Hyun KIM, Seoul (KR)

(21) Appl. No.: 19/360,197

(22) Filed: Oct. 16, 2025

Related U.S. Application Data
(63) Continuation-in-part of application No. 18/076,448, filed on Dec. 7, 2022, now abandoned.

(30) Foreign Application Priority Data
Sep. 22, 2022 (KR) 10-2022-0120001

(51) Int. Cl. G16H 20/30; A61B 5/00

(52) U.S. Cl. CPC G16H 20/30; A61B 5/4542

(57) ABSTRACT

A system for providing a digital therapeutic agent to treat a temporomandibular joint disease of a patient, includes: a disease information identification unit which identifies a temporomandibular joint disease to identify disease information; a therapeutic agent suitability confirmation unit which provides a medical personnel account with at least one digital therapeutic agent information to request the medical personnel account to determine whether the at least one digital therapeutic agent information is suitable for a treatment of the temporomandibular joint disease; a digital therapeutic agent management unit which confirms a validity period of the at least one digital therapeutic agent information to provide a link for installing a digital therapeutic agent and to manage the digital therapeutic agent; and a prescription content providing unit which receives prescription content information for managing the temporomandibular joint disease from the medical personnel account to provide a link for installing a prescription content.

### CROSS-REFERENCE TO RELATED APPLICATOINS

This Application claims the benefit of, and is a continuation of, U.S. Non provisional patent application Ser. No.18/076, 448, filed on Dec. 7, 2022, entitled “SYSTEM FOR PROVIDING DIGITAL THERAPEUTIC AGENT TO TREAT TEMPOROMANDIBULAR JOINT DISEASE OF PATIENT”.

U.S. Ser. No.18/076, 448 claims the priority under 35 U.S.C. § 119 to Korean Patent Application No. 10-2022-0120001, filed on Sep. 22, 2022, to the Korean Intellectual Property Office (KIPO). All of the foregoing applications are hereby incorporated by reference as if fully set forth herein in their entirety.

### TECHNICAL FIELD OF THE INVENTION

The present invention relates to a system for providing a digital therapeutic agent to treat a temporomandibular joint disease of a patient, and more specifically, relates to a technology in which when diagnosis information, which is obtained by diagnosing a temporomandibular joint of a patient, is received from a medical personnel account, a temporomandibular joint disease of the patient is identified through the diagnosis information to identify disease information corresponding to the identified temporomandibular joint disease, when a review of digital therapeutic agent information matched to the disease information is completed by requesting the medical personnel account to review the digital treatment information, an electronic device of the patient is provided with a link for installing the digital therapeutic agent based on digital therapeutic agent information, and simultaneously, a validity period of the digital therapeutic agent is set, and when the patient visits a hospital to receive a treatment from a doctor while treating the temporomandibular joint disease through the digital therapeutic agent, the electronic device is provided with a link for installing a prescription content in order to manage the temporomandibular joint disease to allow the patient to manage the temporomandibular joint disease through the prescription content.

### BACKGROUND OF THE INVENTION

A “digital therapeutic agent” refers to incorporeal software for treating a patient using artificial intelligence, virtual reality (VR), chatbots, games, applications, etc. while undergoing verification of treatment effects through a clinical trial, reviews by regulatory authorities, prescriptions by doctors, and insurance coverage like the ordinary medications. Recently, as the development of digital therapeutic agents, which have emerged as new treatment methods in accordance with the development of information and communications technology (ICT), is in full swing, software itself is directly affecting a disease treatment.

Recently, a medical device market is growing rapidly as a demand for healthcare and aging population are increased worldwide, a digital therapy market, which is a sub-market, also has a bright prospect because the demand for the digital therapy market is increasing. Particularly, as a software technology has a direct effect on diagnosing diseases, monitoring patients, and supporting decisions as well as prevention, management, or treatment of disorders or diseases, digital therapeutic agents are getting more attention. Accordingly, the medical device industries have developed various technologies about digital therapeutic agents for various diseases.

For example, Korean Registered Patent No. 10-2425479 (system and method for creating avatar using user information to provide user information to external metaverse platform and recommending user-customized digital therapeutic agent) discloses a technology that creates an avatar in a virtual world by receiving user's biometric information, activity information, and medical information through a VR device, and calculates a recommendation value for each digital therapeutic agent provided by a plurality of external metaverse platforms based on user information to recommend the digital therapeutic agent to the user.

However, in the above-described prior art, only the technology that calculates the recommendation value about the digital therapeutic agent provided by the plurality of metaverse platforms to provide the digital therapeutic agent to the user is disclosed, a technology is not disclosed in which when diagnosis information, which is obtained by diagnosing a temporomandibular joint of a patient, is received from a medical personnel account, a temporomandibular joint disease of the patient is identified through the diagnosis information to identify disease information corresponding to the identified temporomandibular joint disease, when a review of digital therapeutic agent information matched to the disease information is completed by requesting the medical personnel account to review the digital treatment information, an electronic device of the patient is provided with a link for installing the digital therapeutic agent based on digital therapeutic agent information, and simultaneously, a validity period of the digital therapeutic agent is set, and when the patient visits a hospital to receive a treatment from a doctor while treating the temporomandibular joint disease through the digital therapeutic agent, the electronic device is provided with a link for installing a prescription content in order to manage the temporomandibular joint disease to allow the patient to manage the temporomandibular joint disease through the prescription content. Thus, there is a need for a technology that can solve this problem.

### SUMMARY OF THE INVENTION

Accordingly, an object of the present invention is to provide a system for providing a digital therapeutic agent to treat a temporomandibular joint disease of a patient, which can identify a temporomandibular joint disease of a patient through diagnosis information, which is obtained by diagnosing a temporomandibular joint of the patient, when the diagnosis information is received from a medical personnel account, in order to identify disease information corresponding to the identified temporomandibular joint disease, can provide an electronic device of the patient with a link for installing the digital therapeutic agent based on digital therapeutic agent information when a review of digital therapeutic agent information matched to the disease information is completed by requesting the medical personnel account to review the digital treatment information, can set a validity period of the digital therapeutic agent, and can provide the electronic device with a link for installing a prescription content when the patient visits a hospital to receive a treatment from a doctor while treating the temporomandibular joint disease through the digital therapeutic agent, to allow the patient to manage the temporomandibular joint disease through a prescription content, thereby providing the patient with a digital therapeutic agent recommended based on professional medical knowledge of the doctor, allowing the patient to receive an intermediate treatment from the doctor without visiting the hospital, and providing the patient with the prescription content for managing the temporomandibular joint.

According to one embodiment of the present invention, there is provided a system for providing a digital therapeutic agent to treat a temporomandibular joint disease of a patient, which is implemented in a computing device including one or more processors and one or more memories for storing instructions executable by the processors, includes: a disease information identification unit configured to identify a temporomandibular joint disease of a patient based on diagnosis information, which is obtained by diagnosing a temporomandibular joint of the patient, when the diagnosis information is received from a medical personnel account, in order to identify disease information corresponding to the identified temporomandibular joint disease among a plurality of disease information stored in a pre-stored database; a therapeutic agent suitability confirmation unit configured to provide the medical personnel account with at least one digital therapeutic agent information matched to the identified disease information when the disease information has been identified, in order to request the medical personnel account to determine whether the provided at least one digital therapeutic agent information is suitable for a treatment of the temporomandibular joint disease; a digital therapeutic agent management unit which, when an input indicating that it is suitable for the treatment of the temporomandibular joint disease is received from the medical personnel account in a state in which a function of the therapeutic agent suitability confirmation unit has been performed, confirms a validity period of the at least one digital therapeutic agent information based on the input, in order to provide a link for installing a digital therapeutic agent based on the at least one digital therapeutic agent information in an electronic device of the patient, and to manage the digital therapeutic agent installed in the electronic device such that the digital therapeutic agent is executed during the validity period; and a prescription content providing unit configured to receive prescription content information for managing the temporomandibular joint disease from the medical personnel account when the patient visits a hospital to receive a treatment from a doctor who is a user of the medical personnel account while treating the temporomandibular joint disease through the digital therapeutic agent installed in the electronic device, in order to provide the electronic device with a link for installing a prescription content based on the received prescription content information.

The disease information identification unit may include: a chart information generation unit configured to generate treatment chart information about the patient based on the diagnosis information received from the medical personnel account; and a disease information reflection unit configured to reflect the disease information to the treatment chart information by identifying the temporomandibular joint disease of the patient to identify the disease information corresponding to the identified temporomandibular joint disease among the plurality of disease information stored in the pre-stored database when the treatment chart information has been generated.

The therapeutic agent suitability confirmation unit may include: a therapeutic agent attribute classification unit configured to identify at least one digital therapeutic agent information matched to the identified disease information when a function of the disease information reflection unit has been performed in order to classify the identified at least one digital therapeutic agent information according to an attribute; and a therapeutic agent review request unit configured to provide the medical personnel account with the at least one digital therapeutic agent information classified according to the attribute when the at least one digital therapeutic agent information is classified according to the attribute, in order to request the medical personnel account to determine whether each of the at least one digital therapeutic agent information classified according to the attribute is suitable for the treatment of the temporomandibular joint disease.

The attribute may include: a substitution attribute which is used alone or in combination with an existing treatment for a specific disease to increase a therapeutic effect, as a reference for classification of the at least one digital therapeutic agent information; and a complementary attribute that increases a therapeutic effect of an existing medication.

The digital therapeutic agent management unit may include: a validity period confirmation unit which, when an input indicating that each of the at least one digital therapeutic agent information classified according to the attribute is suitable for the treatment of the temporomandibular joint disease is received from the medical personnel account, confirms the validity period by identifying validity information matched to each of the at least one digital therapeutic agent information classified according to the attribute, based on the input; a membership registration request unit configured to provide the electronic device of the patient with the link for installing the digital therapeutic agent when the validity period has been confirmed in order to allow the patient to sign up for membership to use the digital therapeutic agent when the digital therapeutic agent has been installed through the link; and a validity period allowance unit configured to receive membership information created by the patient when signing up for the membership to use the digital therapeutic agent by performing a function of the membership registration request unit in order to perform a validity period authentication process for the digital therapeutic agent through the received membership information, and to allow the patient to use the digital therapeutic agent during the confirmed validity period when the validity period authentication process has been completed.

The prescription content providing unit may include: an intermediate information reception unit configured to receive, from the electronic device, image information about a chin region captured through the electronic device and abnormality information created based on abnormality of the chin region, when the patient receives treatment of the temporomandibular joint disease through the digital therapeutic agent during the validity period; and a survey information reception unit configured to receive, from the electronic device, symptom survey information created based on a survey provided through the digital therapeutic agent, when the patient receives the treatment of the temporomandibular joint disease through the digital therapeutic agent during the validity period.

The prescription content providing unit may further include: a point identification unit configured to identify, through the abnormality information, a pain point at the chin region of the patient based on the image information, when the image information and the abnormality information have been received; and an intermediate treatment information transmission unit configured to provide the medical personnel account with the received symptom survey information in a state in which the pain point has been identified, in order to allow the medical personnel account to remotely treat a progress of the temporomandibular joint disease corresponding to the pain point to transmit intermediate treatment information based on an intermediate treatment result to the electronic device.

The prescription content providing unit may allow the medical personnel account to create a prescription for managing the temporomandibular joint disease of the patient in order to receive prescription content information based on the created prescription, may identify a prescription content corresponding to the prescription content information among a plurality of prescription contents stored in a pre-stored content database, and may transmit a link for installing the identified prescription content to the electronic device.

The system for providing a digital therapeutic agent to treat a temporomandibular joint disease of a patient according to the present invention can provide a patient with a digital therapeutic agent recommended based on professional medical knowledge of a doctor.

In addition, the system can allow the patient to receive an intermediate treatment from a doctor without visiting a hospital, enable a post-management of patients, and prescribe a prescription content for managing a temporomandibular joint to the patient, such that it is possible for the patient to effectively manage and prevent the temporomandibular joint disease.

### BRIEF DESCRIPTION OF THE DRAWINGS

FIG. 1 is a block diagram of a system for providing a digital therapeutic agent to treat a temporomandibular joint disease of a patient according to one embodiment of the present invention.

FIG. 2 is a block diagram for illustrating a disease information identification unit of the system for providing a digital therapeutic agent to treat a temporomandibular joint disease of a patient according to one embodiment of the present invention.

FIG. 3 is a block diagram for illustrating a therapeutic agent suitability confirmation unit of the system for providing a digital therapeutic agent to treat a temporomandibular joint disease of a patient according to one embodiment of the present invention.

FIG. 4 is a block diagram for illustrating a digital therapeutic agent management unit of the system for providing a digital therapeutic agent to treat a temporomandibular joint disease of a patient according to one embodiment of the present invention.

FIG. 5 is a block diagram for illustrating a prescription content providing unit of the system for providing a digital therapeutic agent to treat a temporomandibular joint disease of a patient according to one embodiment of the present invention.

FIG. 6 is a diagram illustrating an example of a method of providing prescription content to a patient according to an embodiment of the present invention.

FIG. 7 is a diagram for illustrating an example of an internal configuration of a computing device according to one embodiment of the present invention.

### DETAILED DESCRIPTION OF THE INVENTION

Hereinafter, various embodiments and/or aspects will be disclosed with reference to drawings. In the following description, multiple concrete details will be disclosed in order to help general understanding of one or more aspects for the purpose of description. However, it will be recognized by those skilled in the art that the aspect(s) can be executed without the concrete details. In the following disclosure and accompanying drawings, specific exemplary aspects of one or more aspects will be described in detail. However, the aspects are exemplary and some equivalents of various aspects may be used, and the descriptions herein are intended to include both the aspects and equivalents thereto.

It is not intended that any “embodiment”, “example”, “aspect”, “illustration”, and the like used in the specification is preferable or advantageous over any other “embodiment”, “example”, “aspect”, “illustration”, and the like.

Further, the terms “includes” and/or “including” mean that a corresponding feature/or component exists, but it should be appreciated that the terms “include” or “including” mean that presence or addition of one or more other features, components, and/or a group thereof is not excluded.

Further, terms including an ordinal number such as “first” or “second’ may be used for the names of various components, not limiting the components. These expressions are used to distinguish one component from another component. For example, a first component may be referred to as a second component and vice versa without departing the scope of the present invention. The term “and/or” includes a combination of a plurality of related enumerated items or any of the plurality of related enumerated items.

In addition, unless otherwise defined, all terms used herein, including technical or scientific terms, have the same meaning as commonly understood by one of ordinary skill in the art to which the present invention belongs. Terms such as those defined in commonly used dictionaries should be interpreted as having a meaning consistent with the contextual meaning of the related art and should not be interpreted as either ideal or overly formal in meaning unless explicitly defined in the present invention.

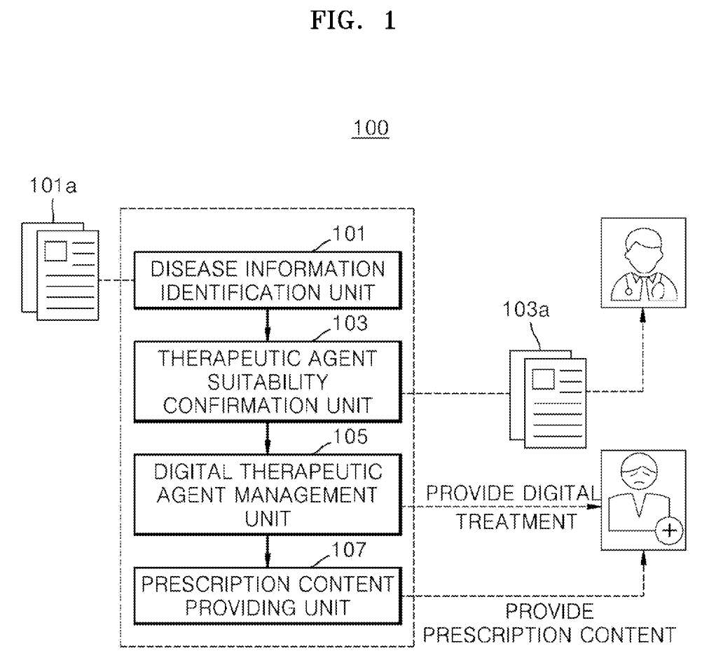

FIG. 1 is a block diagram of a system for providing a digital therapeutic agent to treat a temporomandibular joint disease of a patient according to one embodiment of the present invention.

Referring to FIG. 1, a system 100 for providing a digital therapeutic agent to treat a temporomandibular joint disease of a patient (hereinafter referred to as a digital therapeutic agent providing system) may include a disease information identification unit 101, a therapeutic agent suitability confirmation unit 103, a digital therapeutic agent management unit 105, and a prescription content providing unit 107.

According to one embodiment, when diagnosis information 101a, which is obtained by diagnosing a temporomandibular joint of the patient, is received from a medical personnel account the disease information identification unit 101 may identify a temporomandibular joint disease of the patient based on the diagnosis information 101a to identify disease information corresponding to the identified temporomandibular joint disease among a plurality of disease information stored in a pre-stored database.

According to one embodiment, the diagnosis information 101a may be information including patient's name, patient's date of birth, patient's contact information, patient's age, patient's gender, patient's past medical records, patient's medications, disease name of temporomandibular joint disease of the patient, and symptoms based on the temporomandibular joint disease of the patient.

According to one embodiment, the disease information identification unit 101 may identify the disease name and symptoms of temporomandibular joint disease of the patient based on the received diagnosis information 101a when the diagnosis information 101a is received, to identify the disease information corresponding to the identified disease name of temporomandibular joint disease among the plurality of disease information stored in the pre-stored database.

In connection with the above, the disease information may be information including a disease name of temporomandibular joint disease, a standard disease symptom, a standard prevention guide, a recommended prescription medication, and recommended digital therapeutic agent information. In addition, the pre-stored database may be a database that stores a plurality of disease information, which is information about various diseases.

According to one embodiment, the therapeutic agent suitability confirmation unit 103 may provide the medical personnel account with at least one digital therapeutic agent information 103a matched to the identified disease information when the disease information has been identified by the disease information identification unit 101, to request the medical personnel account to determine whether the provided at least one digital therapeutic agent information 103a is suitable for a treatment of the temporomandibular joint disease (temporomandibular joint disease of the patient).

According to one embodiment, the disease information may include at least one recommended digital therapeutic agent information. Accordingly, the therapeutic agent suitability confirmation unit 103 may identify the recommended digital therapeutic agent information included in the disease information as the at least one digital therapeutic agent information 103a matched to the disease information.

According to one embodiment, the digital therapeutic agent information 103a refers to information based on a digital therapeutic agent classified into artificial intelligence, VR, chatbots, games, applications, etc., and may be information required to install or operate a digital therapeutic agent having a function for treating or managing the temporomandibular joint disease through an electronic device of the patient.

According to one embodiment, the therapeutic agent suitability confirmation unit 103 may provide the medical personnel account with the digital therapeutic agent information 103a to request a doctor who is a user of the medical personnel account to determine whether the digital therapeutic agent based on the provided digital therapeutic agent information 103a is suitable for the treatment of the temporomandibular joint disease.

In this case, the medical personnel account may confirm the provided digital therapeutic agent information 103a to create a professional review based on the suitability for the temporomandibular joint disease of the patient. The professional review may include information about usage of a digital therapeutic agent for the temporomandibular joint disease, usage effect information, detailed usage guide information, information to keep in mind about possible side effects during use of the digital therapeutic agent, validity information (information about a usage validity period of the digital therapeutic agent), validity information for each detailed function (usage validity period of each detailed function included in the digital therapeutic agent), and usage authorization information about the digital therapeutic agent, and the therapeutic agent suitability confirmation unit 103 may receive review confirmation information based on the professional review created by the medical personnel account. Accordingly, the therapeutic agent suitability confirmation unit 103 may confirm, through the received review confirmation information, whether at least one digital therapeutic agent is suitable for the temporomandibular joint disease.

According to one embodiment, in a state in which a function of the therapeutic agent suitability confirmation unit 103 has been performed, when an input (e.g.: review confirmation information) indicating that it is suitable for the treatment of the temporomandibular joint disease is received from the medical personnel account, the digital therapeutic agent management unit 105 may confirm a validity period of at least one digital therapeutic agent information based on the input.

In connection with the above, when the validity period has been confirmed, the digital therapeutic agent management unit 105 may provide the electronic device of the patient with a link for installing the digital therapeutic agent based on at least one digital therapeutic agent information, and may manage the digital therapeutic agent installed in the electronic device such that the digital therapeutic agent is executed during the validity period.

According to one embodiment, when the digital therapeutic agent management unit 105 confirms that at least one digital therapeutic agent is suitable for a treatment of the temporomandibular joint disease of the patient based on the review confirmation information received from the medical personnel account, the digital therapeutic agent management unit 105 may confirm a usage validity period of the digital therapeutic agent based on the received review confirmation information.

According to one embodiment, when the validity period has been confirmed, the digital therapeutic agent management unit 105 may provide the electronic device with a link for installing the digital therapeutic agent in the electronic device of the patient based on the digital therapeutic agent information 103a. In this case, when a user signs up for membership by installing the digital therapeutic agent in the electronic device, the digital therapeutic agent management unit 105 may manage the digital therapeutic agent installed in the electronic device during the validity period through membership information input at the time of signing up for membership.

According to one embodiment, when the patient visits a hospital to receive a treatment from a doctor while treating the temporomandibular joint disease through the digital therapeutic agent installed in the electronic device, the prescription content providing unit 107 may receive prescription content information for managing the temporomandibular joint disease from the medical personnel account to provide the electronic device with a link for installing a prescription content based on the received prescription content information.

According to one embodiment, the prescription content information may be information corresponding to a content in which the doctor treats a status of the temporomandibular joint disease of the patient to allow the patient to manage the temporomandibular joint disease himself/herself when the patient visits the hospital to receive an intermediate treatment for the temporomandibular joint disease from the doctor. Unlike the digital therapeutic agent, the prescription content information is not available on a review by the regulatory authorities and insurance coverage, but may be information based on a content provided to the patient by prescription of the doctor.

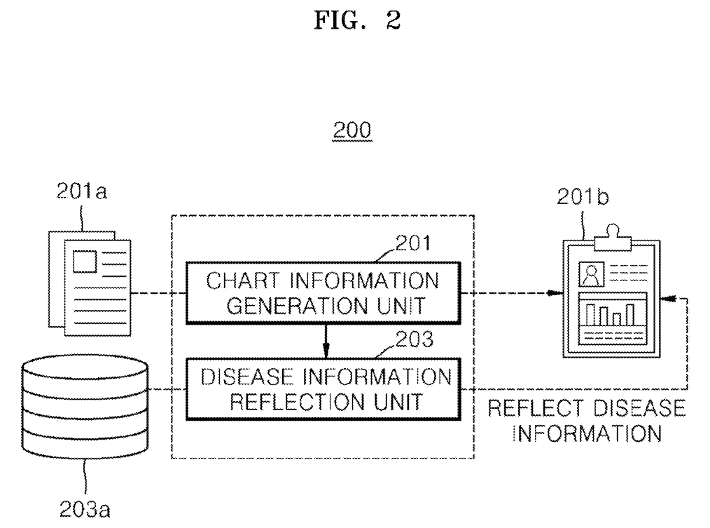

FIG. 2 is a block diagram for illustrating a disease information identification unit of the system for providing a digital therapeutic agent to treat a temporomandibular joint disease of a patient according to one embodiment of the present invention.

Referring to FIG. 2, a system for providing a digital therapeutic agent to treat a temporomandibular joint disease of the patient (e.g.: the system 100 for providing a digital therapeutic agent to treat a temporomandibular joint disease of a patient (hereinafter referred to as a digital therapeutic agent providing system) in FIG. 1) may include a disease information identification unit 200 (e.g.: the disease information identification unit 101 in FIG. 1).

According to one embodiment, when diagnosis information 201a, which is obtained by diagnosing a temporomandibular joint of the patient, is received from a medical personnel account, the disease information identification unit 200 may identify a temporomandibular joint disease of the patient based on the diagnosis information 201a, to identify disease information corresponding to the identified temporomandibular joint disease among a plurality of disease information stored in a pre-stored database 203a.

According to one embodiment, the disease information identification unit 200 may include a chart information generation unit 201 and a disease information reflection unit 203 as detailed components for performing the above-described functions.

According to one embodiment, the chart information generation unit 201 may generate medical chart information 201b about the patient based on diagnosis information 201a received from the medical personnel account.

According to one embodiment, the medical chart information 201b may be information generated when registering and storing information about the patient in a server of the digital therapeutic agent providing system in order to provide the digital therapeutic agent to the patient. The medical chart information 201b may include items based on detailed information included in the diagnosis information 201a, and may be managed and revised by the medical personnel account.

According to one embodiment, the diagnosis information 201a may be information including patient's name, patient's date of birth, patient's contact information, patient's age, patient's gender, patient's past medical records, patient's medications, disease name of temporomandibular joint disease of the patient, and symptoms based on the temporomandibular joint disease of the patient.

According to one embodiment, when the medical chart information 201b has been generated, the disease information reflection unit 203 may identify a temporomandibular joint disease of the patient to identify disease information corresponding to the identified temporomandibular joint disease among a plurality of disease information stored in the pre-stored database 203a and reflect the identified disease information to the medical chart information 201b.

In more detail, the disease information reflection unit 203 may identify a disease name of temporomandibular joint disease of the patient based on the diagnosis information 201a in a state in which the medical chart information 201b has been generated. Accordingly, the disease information reflection unit 203 may identify disease information having a disease name corresponding to the identified disease name among the plurality of disease information stored in the pre-stored database 203a to reflect the identified disease information to the medical chart information 201b. In this case, the information reflected in the medical chart information 201b may reflect a standard disease symptom, a standard prevention guide, a recommended prescription medication, and recommended digital therapeutic agent information.

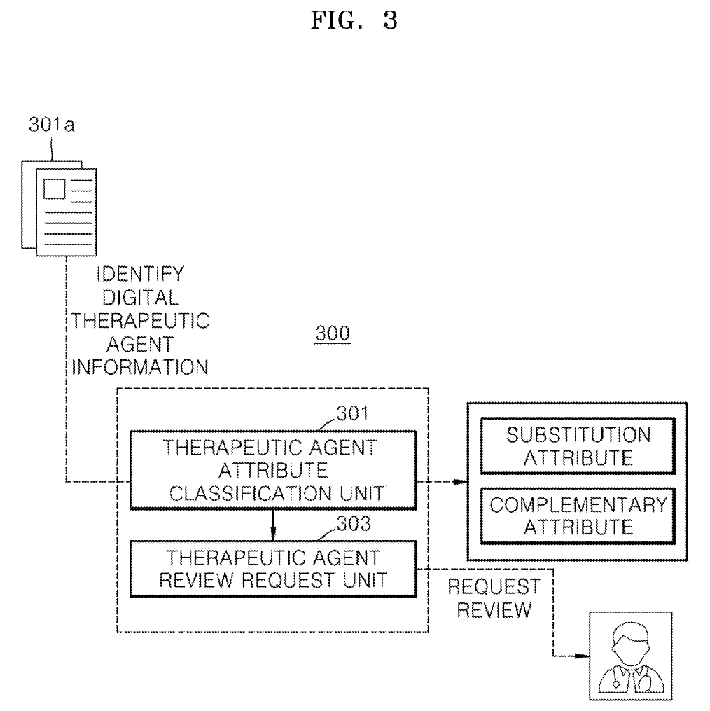

FIG. 3 is a block diagram for illustrating a therapeutic agent suitability confirmation unit of the system for providing a digital therapeutic agent to treat a temporomandibular joint disease of a patient according to one embodiment of the present invention.

Referring to FIG. 3, a system for providing a digital therapeutic agent to treat a temporomandibular joint disease of the patient (e.g.: the system 100 for providing a digital therapeutic agent to treat a temporomandibular joint disease of a patient (hereinafter referred to as a digital therapeutic agent providing system) in FIG. 1) may include a therapeutic agent suitability confirmation unit 300 (e.g.: the therapeutic agent suitability confirmation unit 103 in FIG. 1).

According to one embodiment, when disease information 301a has been identified, the therapeutic agent suitability confirmation unit 300 may provide the medical personnel account with at least one digital therapeutic agent information matched to the identified disease information 301a, to request the medical personnel account to determine whether the provided at least one digital therapeutic agent information is suitable for a treatment of the temporomandibular joint disease.

According to one embodiment, the therapeutic agent suitability confirmation unit 300 may include a therapeutic agent attribute classification unit 301 and a therapeutic agent review request unit 303 as detailed components for performing the above-described functions.

According to one embodiment, when a function of the disease information reflection unit (e.g.: the disease information reflection unit 203 in FIG. 2) has been performed, the therapeutic agent attribute classification unit 301 may identify at least one digital therapeutic agent information matched to the identified disease information 301a to classify the identified at least one digital therapeutic agent information according to an attribute.

In connection with the above, the disease information 301a may be information including a disease name of temporomandibular joint disease, a standard disease symptom, a standard prevention guide, a recommended prescription medication, and recommended digital therapeutic agent information.

In more detail, the therapeutic agent attribute classification unit 301 may identify the recommended digital therapeutic agent information based on the disease information 301a as at least one digital therapeutic agent and confirm an attribute of the identified at least one digital therapeutic agent information.

In connection with the above, the attribute may include a substitution attribute and a complementary attribute as reference information for classifying at least one digital therapeutic agent.

According to one embodiment, the substitution attribute may be an attribute of the digital therapeutic agent, which is used alone or in combination with an existing treatment for a specific disease to increase a therapeutic effect, and the complementary attribute may be an attribute of the digital therapeutic agent, which increases a therapeutic effect of an existing medication.

Accordingly, the therapeutic agent attribute classification unit 301 may confirm attribute information matched to each of the at least one digital therapeutic agent information to identify an attribute of each of the at least one digital therapeutic agent information, and then to classify the at least one digital therapeutic agent information according to the identified attribute.

According to one embodiment, when at least one digital therapeutic agent information is classified according to the attribute, the therapeutic agent review request unit 303 may provide the medical personnel account with at least one digital therapeutic agent information classified according to the attribute, to request the medical personnel account to review whether each of the at least one digital therapeutic agent information classified according to the attribute is suitable for a treatment of the temporomandibular joint disease.

In connection with the above, the therapeutic agent review request unit 303 may provide the medical personnel account with a list of the at least one digital therapeutic agent information classified according to the attribute. The list refers to an interface that outputs at least one digital therapeutic agent information classified according to the attribute, and may be an interface that is utilized by the medical personnel account. In this case, at least one digital therapeutic agent information recommended on the list may be output while being sorted in the order in which it is most frequently selected by other medical personnel accounts.

In this case, when the medical personnel account selects one of at least one digital therapeutic agent information output for each attribute from the list, disease information corresponding to the selected digital therapeutic agent information, as well as guide information about detailed functions included in the digital therapeutic agent information, manufacturer information, manufacturer contact information, medical insurance information, items for creating professional reviews may be output.

Accordingly, when the professional reviews are created as the medical personnel account selects the at least one digital therapeutic agent information output from the list, the therapeutic agent review request unit 303 may generate review confirmation information based on the created professional reviews.

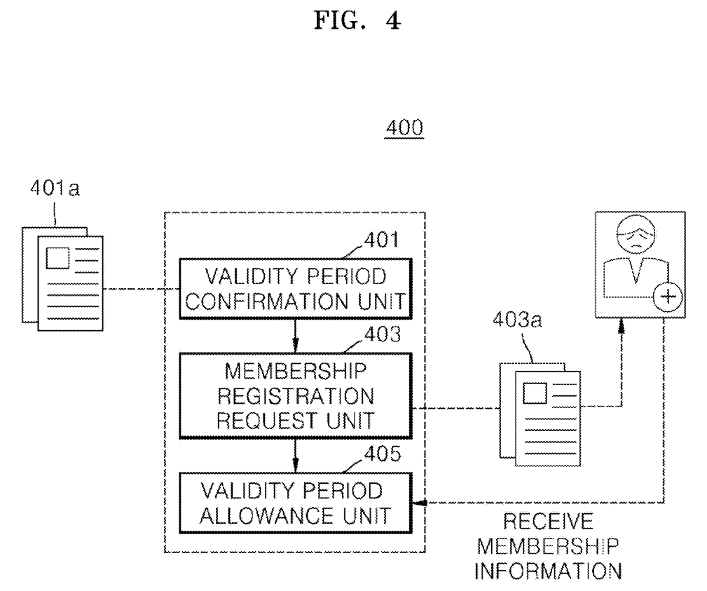

FIG. 4 is a block diagram for illustrating a digital therapeutic agent management unit of the system for providing a digital therapeutic agent to treat a temporomandibular joint disease of a patient according to one embodiment of the present invention.

Referring to FIG. 4, a system for providing a digital therapeutic agent to treat a temporomandibular joint disease of the patient (e.g.: the system 100 for providing a digital therapeutic agent to treat a temporomandibular joint disease of a patient (hereinafter referred to as a digital therapeutic agent providing system) in FIG. 1) may include a digital therapeutic agent management unit 400 (e.g.: the digital therapeutic agent management unit 105 in FIG. 1).

According to one embodiment, in a state in which the function of the therapeutic agent suitability confirmation unit (e.g.: the therapeutic agent suitability confirmation unit 103 in FIG. 1) has been performed, when an input indicating that it is suitable for the treatment of the temporomandibular joint disease is received from the medical personnel account, the digital therapeutic agent management unit 400 may confirm a validity period of the at least one digital therapeutic agent information based on the input, to provide a link for installing, in the electronic device of the patient, the digital therapeutic agent based on the at least one digital therapeutic agent information and to manage the digital therapeutic agent installed in the electronic device such that the digital therapeutic agent is executed during the validity period.

According to one embodiment, the digital therapeutic agent management unit 400 may include a validity period confirmation unit 401, a membership registration request unit 403, and a validity period allowance unit 405 as detailed components for performing the above-described functions.

According to one embodiment, when an input indicating that each of the at least one digital therapeutic agent information classified according to the attribute is suitable for the treatment of the temporomandibular joint disease (temporomandibular joint disease of the patient) is received from the medical personnel account, the validity period confirmation unit 401 may identify validity information matched to each of the at least one digital therapeutic agent information classified according to the attribute based on the input to confirm a validity period.

In more detail, the validity period confirmation unit 401 may confirm the validity period of each of the at least one digital therapeutic agent information classified according to the attribute based on review confirmation information 401a received from the medical personnel account, thereby confirming the validity period of at least one digital therapeutic agent information classified according to the attribute. In this case, information with the validity information matched (included) among the at least one digital therapeutic agent classified according to the attribute may be information that is allowed to be provided to the patient by the medical personnel account.

According to one embodiment, when the validity period has been confirmed, the membership registration request unit 403 may provide the electronic device of the patient with a link 403a for installing a digital therapeutic agent corresponding to at least one digital therapeutic agent information classified according to the attribute (digital therapeutic agent information with the validity information matched), to request the patient to sign up for membership to use the digital therapeutic agent when the digital therapeutic agent has been installed through the link 403a.

According to one embodiment, the membership registration request unit 403 may provide the patient with the link 403a, together with installation guide information for guiding installation of the digital therapeutic agent in the electronic device through the link 403a and an invitation code that the patient needs to be input when signing up for membership to use the digital therapeutic agent.

According to one embodiment, the validity period allowance unit 405 may receive membership information created when the patient signs up for membership to use the digital therapeutic agent by performing the function of the membership registration request unit 403. The validity period allowance unit 405 may perform a validity period authentication process for the digital therapeutic agent through the received membership information to allow the patient to use the digital therapeutic agent during the confirmed validity period when the validity period authentication process has been completed.

According to one embodiment, the validity period authentication process may be an authentication process for allowing the patient who has signed up for membership to use the digital therapeutic agent installed in the electronic device during a specified period (validity period).

In this case, when the authentication process for the digital therapeutic agent installed in the electronic device is performed, the validity period allowance unit 405 may confirm whether detailed information included in the membership information (e.g.: patient's name, patient's date of birth, patient's contact information, patient's age, and patient's gender) matches some of detailed information included in the medical chart information (e.g.: patient's name, patient's date of birth, patient's contact information, patient's age, and patient's gender input in the medical chart information).

In connection with the above, when the detailed information included in the membership information matches the detailed information included in the medical chart information, the validity period allowance unit 405 requests the patient to additionally input the disease name of current temporomandibular joint disease of the patient and the invitation code, and when both the disease name and the invitation code input by the patient match, the validity period allowance unit 405 may assign authorization, which is set to use the digital therapeutic agent, to an account of the patient. In this case, the assigned authorization may be authorization maintained for the validity period (authorization in which the patient may use the digital therapeutic agent during the validity period). In addition, the validity period allowance unit 405 may confirm the validity information about each of the detailed functions included in the digital therapeutic agent to activate or inactivate the detailed functions of the digital therapeutic agent according to the validity period of each of the confirmed detailed functions.

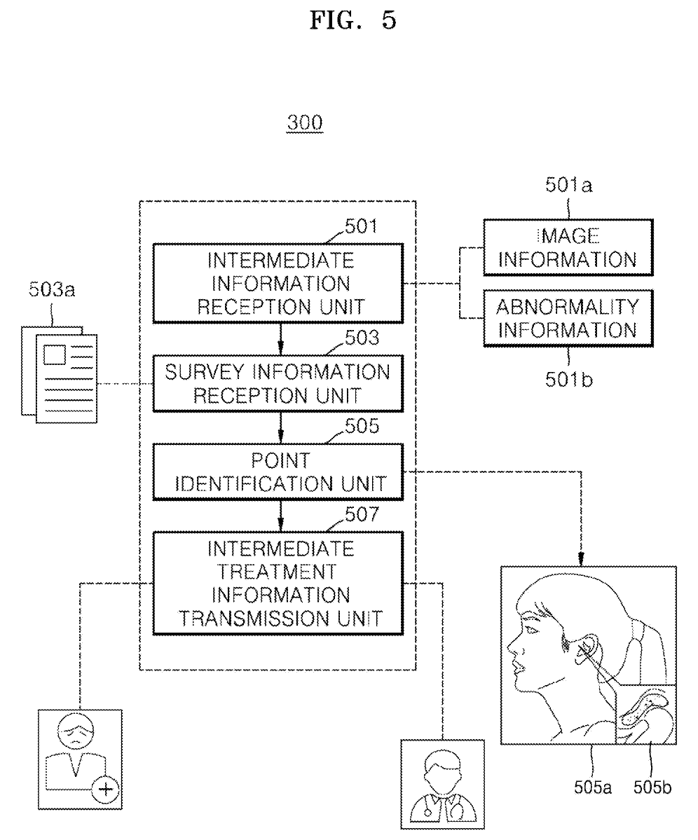

FIG. 5 is a block diagram for illustrating a prescription content providing unit of the system for providing a digital therapeutic agent to treat a temporomandibular joint disease of a patient according to one embodiment of the present invention. Referring to FIG. 5, a system for providing a digital therapeutic agent to treat a temporomandibular joint disease of the patient (e.g.: the system 100 for providing a digital therapeutic agent to treat a temporomandibular joint disease of a patient (hereinafter referred to as a digital therapeutic agent providing system) in FIG. 1) may include a prescription content providing unit 500 (e.g.: the prescription content providing unit 107 in FIG. 1).

According to one embodiment, when the patient visits the hospital to receive a treatment from the doctor who is a user of the medical personnel account while treating the temporomandibular joint disease through the digital therapeutic agent installed in the electronic device, the prescription content providing unit 500 may receive prescription content information for managing the temporomandibular joint disease from the medical personnel account to provide the electronic device with a link for installing a prescription content based on the received prescription content information.

In more detail, when the patient visits the hospital to receive a treatment from the doctor who is a user of the medical personnel account, the prescription content providing unit 500 may allow the medical personnel account to create a prescription for managing the temporomandibular joint disease of the patient to generate prescription content information based on the created prescription.

In connection with the above, the prescription content information may be information corresponding to a content in which the doctor treats a status of the temporomandibular joint disease of the patient to allow the patient to manage the temporomandibular joint disease himself/herself when the patient visits the hospital and receives an intermediate treatment for the temporomandibular joint disease from the doctor. That is, the prescription may be created by the medical personnel account to provide the patient with at least one of a plurality of prescription contents stored in a pre-stored content database.

According to one embodiment, when the prescription content information has been generated, the prescription content providing unit 500 may identify a prescription content corresponding to the prescription content information among the plurality of prescription contents stored in the pre-stored content container to transmit a link for installing the identified prescription content to the electronic device. In this case, the prescription content may include exercises for alleviating a progress of temporomandibular joint disease or reducing pain, images, videos, and guidance guides for countermeasures, etc

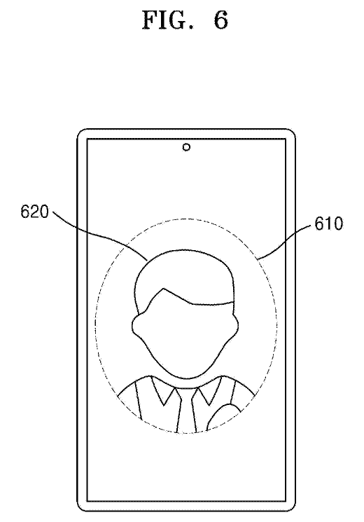

More specifically, the exercises included in the prescription content may refer to contents which provide the patient with a list of exercises which are helpful for the health of a temporomandibular joint and help the patient perform the exercises selected by the patient. Meanwhile, a specific method of providing the patient with the exercises for alleviating a progress of temporomandibular joint disease or reducing pain as a prescription content by the prescription content providing unit 500 is described below with reference to FIG. 6.

According to one embodiment, the prescription content providing unit 500 may include a function of allowing the medical personnel account to remotely treat the patient in addition to the above-described functions.

According to one embodiment, the prescription content providing unit 500 may include an intermediate information reception unit 501, a survey information reception unit 503, a point identification unit 505, and an intermediate treatment information transmission unit 507 as detailed components for performing the above-described functions.

According to one embodiment, the intermediate information reception unit 501 may receive, from the electronic device, image information 501a about a chin region captured through the electronic device and abnormality information 501b created based on abnormality of the chin region, when the patient receives a treatment of the temporomandibular joint disease through the digital therapeutic agent during the validity period.

According to one embodiment, the image information 501a may be an image for a chin region of the patient, captured by the patient at various angles through a camera of the electronic device. In this case, the patient may display a pain object on one region of the chin region in the image where the pain occurs.

According to one embodiment, the abnormality information 501b may be information generated as the patient creates the feeling of pain, the cycle of pain, the date and time of pain onset, and the requests for the region in which pain occurs. In this case, the abnormality information 501b may be generated based on contents created by the patient corresponding to the pain object.

According to one embodiment, when the patient receives the treatment of temporomandibular joint disease through the digital therapeutic agent during the validity period, the survey information reception unit 503 may receive, from the electronic device, symptom survey information 503a created based on a survey provided through the digital therapeutic agent.

According to one embodiment, the symptom survey information 503a may be information generated as the patient creates a survey based on items that the doctor intensively asks the patient who suffers from the temporomandibular joint disease for questions (e.g.: status compared to the last treatment, whether the patient normally takes medications, whether the patient uses well the specific functions of the digital therapeutic agent).

According to one embodiment, the point identification unit 505 may identify, through the abnormality information 501b, a pain point at the chin region of the patient based on the image information 501a, when the image information 501a and the abnormality information 501b have been received. In this case, the pain point may refer to a region in which the pain object is located in the image.

In connection with the above, the point identification unit 505 may identify the pain object in the image based on the image information 501a, and confirm the feeling of pain, the cycle of pain, the date and time of pain onset, and the requests in the region in which the identified pain object is located through the abnormality information 501b. In this case, the point identification unit 505 may provide the medical personnel account with the pain object identified in the image based on the image information 501a and the feeling of pain, the cycle of pain, the date and time of pain onset, and the requests in the region in which the identified pain object is located.

In an embodiment, the point identification unit 505 may extract at least one region where the patient feels pain from an entire image 505a of the patient based on the image information 501a and the abnormality information 501b. In more detail, the point identification unit 505 may obtain the image information 501a of the patient, and based on the obtained image information 501a of the patient and the abnormality information 501b of the patient, the unit may extract a region where the patient feels pain from the entire image 505a of the patient. That is, the point identification unit 505 may extract the region where the patient feels pain from the entire image 505a of the patient based on the abnormality information 501b of the patient.

In an embodiment, the point identification unit 505 may additionally extract an image 505b of a region where the patient feels pain from the entire image 505a of the patient. That is, the point identification unit 505 may identify the region where the patient feels pain by using the image information 501a of the patient and the abnormality information 501b of the patient, and the unit may extract the image of the region from the entire image 505a of the patient. In addition, the point identification unit 505 may obtain an input from the patient to specify a more precise region where the patient feels pain by providing the patient with the image 505b of the region where the patient feels pain. That is, the image 505b of the region where the patient feels pain may be provided to the patient through a display or an interface included in the electronic device, and the point identification unit 505 may obtain an input from the patient through the display or interface to specify a more precise region where the patient feels pain.

In an embodiment, the point identification unit 505 may extract chin feature points of the patient from the image information 501a of the patient. In addition, the point identification unit 505 may identify the region where the patient feels pain by using the extracted chin feature points and the abnormality information 501b of the patient.

Accordingly, in case that there is no input regarding the region where the patient feels pain or in case that the patient feels pain and does not know the precise region of the pain, the point identification unit 505 may extract the region of the pain from the entire image 505a of the patient and provide information about the feeling of pain, the region of pain, or the like to the medical personnel account.

According to one embodiment, the intermediate treatment information transmission unit 507 may provide the medical personnel account with the received symptom survey information 503a in a state in which the pain point has been identified, to allow the medical personnel account to remotely treat a progress of the temporomandibular joint disease corresponding to the pain point to transmit the intermediate treatment information based on an intermediate treatment result to the electronic device.

Meanwhile, the intermediate treatment information transmission unit 507 may directly provide the abnormality information 501b obtained from the patient to the medical personnel account.

In an embodiment, the intermediate information reception unit 501 may provide record items for abnormality information to the patient and obtain an input from the patient regarding at least one region where the patient feels pain, the feeling of pain, the cycle of pain, the date and time of pain onset, the degree of pain, and the requests that correspond to the record items for abnormality information.

In an embodiment, the intermediate information reception unit 501 may obtain an input from the patient regarding the region of each of the left and right body parts of the patient where pain occurs. At this time, the intermediate information reception unit 501 may obtain an input from the patient regarding at least one of a point or a region where the patient feels pain. In other words, the intermediate information reception unit 501 may obtain an input from the patient regarding a specific part or region where the patient feels pain.

In an embodiment, the intermediate information reception unit 501 may obtain an input from the patient regarding a specific point where the patient feels pain. In another embodiment, the intermediate information reception unit 501 may obtain an input from the patient regarding a specific region where the patient feels pain, and the specific region may be determined by a drag input of the patient or an input for selecting a plurality of points. Here, the drag input may refer to an input in which the patient moves while directly touching the display or interface included in the electronic device.

In an embodiment, the intermediate information reception unit 501 may obtain an input from the patient regarding a region where the patient feels pain and then obtain an input regarding the degree of pain in the region. At this time, the degree of pain may be entered as a score from 0 to 10, with a lower score indicating less intensity and a higher score indicating greater pain. Additionally, the degree of pain may be input not only as a score, but also as a grade of high/medium/low, a text, or the like, but this is not limited thereto.

Additionally, the intermediate information reception unit 501 may obtain an input from the patient regarding a situation in which the patient feels pain in the region.

In an embodiment, the intermediate information reception unit 501 may obtain only an input related to pain of the patient, the input satisfying a predetermined condition.

In an embodiment, the intermediate information reception unit 501 may determine whether inputs obtained from the patient, including an input regarding at least one region where the patient feels pain and an input regarding the degree of pain, satisfy a predetermined condition. At this time, the predetermined condition may include a condition of obtaining a touch input or drag input for selecting at least one region where the patient feels pain.

Accordingly, the intermediate information reception unit 501 may determine whether an input for selecting at least one region where the patient feels pain is a touch input or drag input, and based on the fact that the input is a touch input or drag input, the unit may determine that the condition for obtaining a touch input or drag input is satisfied.

In an embodiment, the intermediate information reception unit 501 may obtain an input determined to satisfy a predetermined condition.

In an embodiment, the intermediate information reception unit 501 may obtain an input regarding the degree of pain corresponding to the region selected based on determining that the input for selecting the region where the patient feels pain satisfies the condition for obtaining a touch input or drag input.

Accordingly, the intermediate information reception unit 501 may provide the patient with an opportunity to recognize his or her own pain by obtaining only an input which satisfies a predetermined condition during the process of inputting a region where the patient feels pain and the degree of pain in the region.

According to one embodiment, the doctor who is a user of the medical personnel account may remotely treat the progress of the temporomandibular joint disease of the patient through the pain object identified in the image based on the image information 501a, the feeling of pain, the cycle of pain, the date and time of pain onset, and the requests in the region in which the identified pain object is located, which are received from the point identification unit 505, and the symptom survey information 503a.

According to another embodiment, a doctor who is a user of the medical personnel account may remotely diagnose the progress of the temporomandibular joint disease suffered by the patient through the abnormality information 501b such as at least one region where the patient feels pain, the feeling of pain, the cycle of pain, the date and time of pain onset, the degree of pain, and the requests, the information provided by the intermediate treatment information transmission unit 507.

Accordingly, the intermediate treatment information transmission unit 507 may generate intermediate treatment information based on a remote treatment result (intermediate treatment result) of the medical personnel account to transmit the intermediate treatment information to the electronic device of the patient.

In this case, the intermediate treatment information may include a doctor's professional opinion on the progress of the temporomandibular joint disease of the patient, detailed functions of the digital therapeutic agent recommended according to the progress of the disease, usage guide methods of the detailed functions, a link for installing other digital therapeutic agents according to the progress of the disease, and a link of other prescription contents according to the progress of the disease.

FIG. 6 is a drawing illustrating an example of a method for providing a prescription content to a patient according to an embodiment of the disclosure.

Referring to FIG. 6, a prescription content providing unit may provide the patient with exercises included in the prescription content. At this time, the prescription content providing unit may refer to the prescription content providing unit 107 or the prescription content providing unit 500 described above with reference to FIGS. 1 and 5.

In an embodiment, the prescription content providing unit may provide a list of exercises and provide exercise image information or patient image information so that the patient may perform an exercise selected from the provided list of exercises.

In an embodiment, the prescription content providing unit may provide the patient with at least one of an explanatory video of an exercise, an explanatory text of an exercise, and an explanatory voice of an exercise.

In an embodiment, the prescription content providing unit may provide exercises to the patient and provide feedback data to the patient based on data from the patient performing the exercises. At this time, the prescription content providing unit may determine whether the patient is performing the exercises correctly based on an image 620 of the patient.

In an embodiment, the prescription content providing unit may determine whether the image 620 of the patient is included in a predetermined frame 610.

In an embodiment, the prescription content providing unit may determine whether a face of the patient included in the image 620 of the patient performing the exercises is included in the predetermined frame 610. In addition, the prescription content providing unit may determine the degree to which the image 620 of the patient matches the predetermined frame 610, and, based on a determination result, determine whether the patient is performing the exercises. In addition, the prescription content providing unit may determine whether the image 620 of the patient is within a preset range and, based on a determination result, determine whether the patient is performing the exercises.

In an embodiment, the prescription content providing unit may input the image 620 of the patient into a face detection model as input data of the face detection model and obtain a face detection result of the patient as output data of the face detection model.

At this time, the face detection model may be an artificial intelligence model trained to output a result of detecting a face from a predetermined image by using the predetermined image as learning data. More specifically, the face detection model may be an artificial intelligence model trained to extract at least one facial feature point (e.g. an eye, a nose, a mouth, an ear) from a predetermined image which has been input and to detect a face from the predetermined image based on the extracted facial feature point.

Accordingly, the prescription content providing unit may determine whether the detected face of the patient is included in the predetermined frame 610.

In an embodiment, the prescription content providing unit may determine that the face of the patient is included in the predetermined frame 610, in case that the detected face of the patient is included in a preset area or more within the predetermined frame 610. Here, the preset area may be 80% of the area of the predetermined frame 610, but this is not limited thereto.

In an embodiment, the prescription content providing unit may determine the degree to which the detected face of the patient matches the predetermined frame 610. Additionally, the prescription content providing unit may determine whether the detected face of the user is included within a preset range.

In an embodiment, the prescription content providing unit may determine whether the patient is performing the exercises based on a determined result.

In an embodiment, in case that the prescription content providing unit determines that the detected face of the patient is included in the predetermined frame 610, the unit may determine that the patient is performing the exercises. In another embodiment, in case that the prescription content providing unit determines that the detected face of the patient is not included in the predetermined frame 610, the unit may determine that the patient is not performing the exercises.

In an embodiment, the prescription content providing unit may determine that the patient is performing exercises based on determining that the face of the patient and the predetermined frame 610 match each other by a preset value or more. Here, the preset value may be 80%, but this is not limited thereto. Additionally, the prescription content providing unit may determine that the patient is performing the exercises based on the determination that the detected face of the patient is within a preset range. Here, the preset range may be a parameter for determining whether the patient is performing the exercises separately from the predetermined frame 610 and refer to a portion of the total area of the display or interface included in the electronic device.

Therefore, in case that it is determined that the patient is not performing the exercises, the prescription content providing unit may provide a notification to the patient.

In an embodiment, in case that the face of the patient moves out of the predetermined frame 610 or in case that a gaze of the patient moves away from the display or interface included in the electronic device, the prescription content providing unit may temporarily stop providing the exercises and provide a notification to the patient.

In an embodiment, the prescription content providing unit may determine whether the face of the patient is included in the predetermined frame 610 again within a preset time period after the provision of the exercises has been stopped, or whether the gaze of the patient is directed toward the display or interface included in the electronic device.

In an embodiment, the prescription content providing unit may provide the exercises to the patient again and provide an alarm based on a result of determining that the face of the patient is included in the predetermined frame 610 again within the preset time period while the provision of the exercises has been stopped. In addition, the prescription content providing unit may provide the exercises to the patient again and provide an alarm based on a result of determining that the gaze of the patient is directed toward the display or interface included in the device again within the preset time period while the provision of the exercises has been stopped.

In another embodiment, the prescription content providing unit may terminate the provision of the exercises and provide an alarm based on a result of determining that the face of the patient is not included in the predetermined frame 610 again within the preset time period while the provision of the exercises has been stopped. In addition, the prescription content providing unit may terminate the provision of the exercises and provide an alarm based on a result of determining that the gaze of the patient is not directed toward the display or interface included in the electronic device again within the preset time period while the provision of the exercises has been stopped. Meanwhile, the prescription content providing unit may track the gaze of the patient based on the image 620 of the patient.

In an embodiment, the prescription content providing unit may track the gaze of the patient by using the image of the patient performing the exercises.

In an embodiment, the prescription content providing unit may input the image 620 of the patient into a gaze tracking model as input data of the gaze tracking model and obtain a gaze tracking result of the patient as output data of the gaze tracking model.

In an embodiment, the gaze tracking model may be an artificial intelligence model trained to output a result of tracking the gaze of a person included in a predetermined image by using the predetermined image as learning data. More specifically, the gaze tracking model may be an artificial intelligence model trained to extract at least one facial feature point (e.g. an eye, a nose, a mouth, an ear) from a predetermined image which has been input and track a gaze from the predetermined image based on the extracted facial feature point.

In an embodiment, the prescription content providing unit may determine whether the patient is performing the exercises based on the gaze of the patient.

In an embodiment, the prescription content providing unit may determine whether the gaze of the patient is directed toward the display or interface included in the electronic device based on the tracked gaze of the patient.

In an embodiment, in case that the prescription content providing unit determines that the gaze of the patient is directed toward the display or interface included in the electronic device, the unit may determine that the patient is performing the exercises. In another embodiment, in case that the prescription content providing unit determines that the gaze of the patient is not directed toward the display or interface included in the electronic device, the unit may determine that the patient is not performing the exercises.

In an embodiment, the prescription content providing unit may obtain condition information of an eye area of the patient included in the image 620 of the patient.

In an embodiment, the prescription content providing unit may obtain the condition information of the eye area of the patient by using the image of the patient performing the exercises.

In an embodiment, the prescription content providing unit may input the image of the patient into an information extraction model as input data of the information extraction model and obtain the condition information of the eye area of the patient as output data of the information extraction model.

In an embodiment, the information extraction model may be an artificial intelligence model trained to extract the condition information of the eye area, such as eye movement, pupil size, and eyelid position, from a predetermined image by using the predetermined image as learning data and to output a result of extracting the information. More specifically, the information extraction model may be an artificial intelligence model trained to extract an eye feature point from a predetermined image which has been input image and to extract the condition information of the eye area based on the extracted eye feature point.

Accordingly, the prescription content providing unit may extract an eye feature point of the patient from an image of the patient performing exercises and extract condition information of the eye area.

In an embodiment, the prescription content providing unit may determine whether the patient is capable of performing the exercises based on the condition information of the eye area.

In an embodiment, the prescription content providing unit may determine whether the patient is in a state where he or she may perform the exercises based on a pupil size, eye tremors, an eyelid position, or the like of the patient that are included in the condition information of the eye area.

In an embodiment, in case that the pupil size of the patient is smaller than the average for a normal person, the eye tremors of the patient are greater than the average for a normal person, and the eyelid position is lower than usual, the prescription content providing unit may determine that the patient is in a state where he or she is unable to perform the exercises. In another embodiment, in case that the pupil size of the patient is similar to or larger than the average for a normal person, the eye tremors of the patient are similar to or smaller than the average for a normal person, and the eyelid position is similar to or higher than usual, the prescription content providing unit may determine that the patient is in a state where he or she may perform the exercises.

Accordingly, in case that the prescription content providing unit determines that the patient is unable to perform the exercises, the unit may provide a notification to the patient to stop performing the exercises.

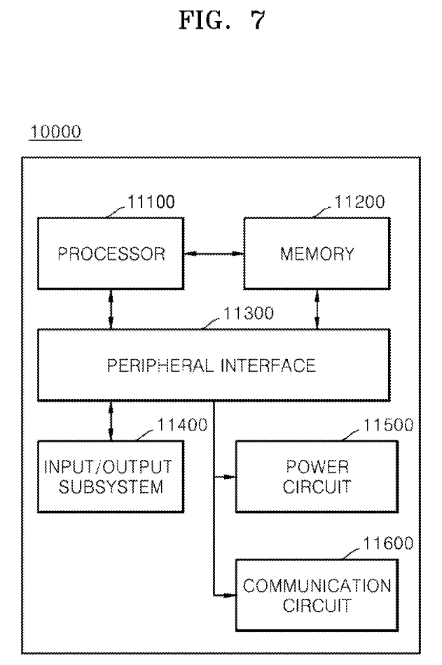

FIG. 7 is a diagram for illustrating an example of an internal configuration of a computing device according to one embodiment of the present invention.

FIG. 7 illustrates an example of an internal configuration of a computing device according to one embodiment of the present invention, and in the following descriptions, descriptions for unnecessary embodiments overlapping with the above-described descriptions of FIGS. 1 to 6 will be omitted.

As shown in FIG. 7, a computing device 10000 may at least include at least one processor 11100, a memory 11200, a peripheral interface 11300, an input/output (I/O) subsystem 11400, a power circuit 11500, and a communication circuit 11600. In this case, the computing device 10000 may correspond to a user terminal A connected to a tactile interface device or a computing device B described above.

The memory 11200 may include, for example, a high-speed random access memory, a magnetic disk, static random access memory (SRAM), a dynamic RAM (DRAM), read only memory (ROM), a flash memory, and a non-volatile memory. The memory 11200 may include a software module, an instruction set, or a variety of data as required for operation of the computing device 10000.

In this case, access from another component, such as the processor 11100 and the peripheral interface 11300, to the memory 11200 may be controlled by the processor 11100.

The peripheral interface 11300 may couple an input and/or output peripheral device of the computing device 10000 to the processor 11100 and the memory 11200. The processor 1410 may perform a variety of functions for the computing device 10000 and may process data by executing the software module or the instruction set stored in the memory 11200.

The input/output subsystem 11400 may couple various input/output peripheral devices to the peripheral interfaces 11300. For example, the input/output subsystem 11400 may include a controller for coupling the peripheral devices, such as a monitor, a keyboard, a mouse, a printer, and a touch screen or a sensor as necessary, to the peripheral interface 11300. According to another aspect, the input/output peripheral devices may be coupled to the peripheral interface 11300 without using the input/output subsystem 11400.

The power circuit 112500 may supply power to all of or a part of the components of a terminal. For example, the power circuit 11500 may include a power management system, at least one power source such as a battery or alternating circuit (AC), a charging system, a power failure detection circuit, a power converter or inverter, a power status indicator, or other components for generating, managing and distributing power.

The communication circuit 11600 may communicate with other computing devices using at least one external port.

Alternatively, as described above, the communication circuit 11600 may communicate with other computing devices by including a radio frequency (RF) circuit and thereby transmitting and receiving an RF signal known as an electromagnetic signal.

The embodiment of FIG. 6 is merely an example of the computing device 10000, and the computing device 10000 may have a configuration or an arrangement in which a part of the components illustrated in FIG. 6 has been omitted, or it may further include components that are not illustrated in FIG. 6 or it may include the coupling of two or more components. For example, a computing device for a communication terminal of a mobile environment may further include a touch screen, a sensor, and the like, in addition to the components of FIG. 6. A circuit for RF communication using a variety of communication methods (wireless fidelity (Wi-Fi), 3rd generation (3G), long term evolution (LTE), Bluetooth, near field communication (NFC), and ZigBee) may be included in the communication circuit 11600. Components includable in the computing device 10000 may be implemented as hardware that includes an integrated circuit specified for at least one signal processing or application, software, or a combination of hardware and software.

The methods according to the embodiment of the present invention may be implemented in the form of a program instruction that may be executed through various computing devices, thereby being recorded in computer-readable media. In particular, the program according to the present embodiment may be configured with a PC-based program or a mobile terminal dedicated application. An application to which the present invention is applied may be installed in a user terminal through a file provided by a file distribution system. For example, the file distribution system may include a file transmission unit (not shown) that transmits files according to a request of the user terminal.

The device described above may be implemented as hardware components, software components, and/or a combination of the hardware components and the software components. For example, the device and the components described above may be implemented using one or more general-purpose or special purpose computers, such as, for example, a processor, a controller and an arithmetic logic unit (ALU), a digital signal processor, a microcomputer, a field programmable gate array (PGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of the processing device is used as singular; however, one skilled in the art will be appreciated that the processing device may include multiple processing elements and multiple types of processing elements. For example, the processing device may include multiple processors or a processor and a controller. In addition, different processing configurations are possible, such as parallel processors.

The software may include a computer program, a piece of code, an instruction, or some combination thereof, for independently or collectively instructing or configuring the processing device to operate as desired. Software and/or data may be embodied permanently or temporarily in any type of machine, component, physical or virtual equipment, computer storage medium or device, or in a propagated signal wave capable of providing instructions or data to or being interpreted by the processing device. The software may also be distributed over network-connected computing devices so that the software is stored and executed in a distributed manner. The software and data may be stored by one or more computer-readable recording media.

The above-described embodiments of the present invention may be recorded in computer-readable media including program instructions to implement various operations embodied by a computer. The media may also include, alone or in combination with the program instructions, data files, data structures, and the like. The program instructions recorded on the media may be those specially designed and constructed for the purposes of the embodiments, or they may be of the kind well-known and available to those having skill in the computer software arts. Examples of computer-readable recording media include magnetic media such as hard disks, floppy disks, and magnetic tape; optical media such as CD ROM disks and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations of the above-described embodiments of the present invention, or vice versa.

As described above, although the embodiments have been described with reference to limited examples and drawings, it will be apparent to one of ordinary skill in the art that various modifications and variations may be made in the above descriptions. For example, suitable results may be achieved if the described techniques are performed in a different order, and/or if components in a described system, structure, device, or circuit are combined in a different manner as described above, and/or replaced or supplemented by other components or their equivalents. Therefore, other implementations, other embodiments, and equivalents of the claims are within the scope of the following claims.

What is claimed is:

1. A system for providing a digital therapeutic agent to treat a temporomandibular joint disease of a patient, which is implemented in a computing device including one or more processors and one or more memories for storing instructions executable by the processors, the system comprising:
a disease information identification device configured to identify a temporomandibular joint disease of a patient based on diagnosis information, which is obtained by diagnosing a temporomandibular joint of the patient, when the diagnosis information is received from a medical personnel account, in order to identify disease information corresponding to the identified temporomandibular joint disease among a plurality of disease information stored in a pre-stored database;
a therapeutic agent suitability confirmation device configured to provide the medical personnel account with at least one digital therapeutic agent information matched to the identified disease information when the disease information has been identified, in order to request the medical personnel account to determine whether the provided at least one digital therapeutic agent information is suitable for a treatment of the temporomandibular joint disease;
a digital therapeutic agent management device which, when an input indicating that it is suitable for the treatment of the temporomandibular joint disease is received from the medical personnel account in a state in which a function of the therapeutic agent suitability confirmation device has been performed, confirms a validity period of the at least one digital therapeutic agent information based on the input, in order to provide a link for installing a digital therapeutic agent based on the at least one digital therapeutic agent information in an electronic device of the patient, and to manage the digital therapeutic agent installed in the electronic device such that the digital therapeutic agent is executed during the validity period; and
a prescription content providing device configured to receive prescription content information for managing the temporomandibular joint disease from the medical personnel account when the patient visits a hospital to receive a treatment from a doctor who is a user of the medical personnel account while treating the temporomandibular joint disease through the digital therapeutic agent installed in the electronic device, in order to provide the electronic device with a link for installing a prescription content based on the received prescription content information,
wherein the prescription content providing device includes a point identification device configured to identify a region where the patient feels pain by using the image information of the patient and the abnormality information of the patient, to extract a image of the region where the patient feels pain from the entire image of the patient and to obtain an input from the patient to specify a more precise region where the patient feels pain through an electronic device by providing the patient with the image of the region where the patient feels pain through the electronic device.

2. The system of claim 1, wherein the disease information identification device includes:
a chart information generation device configured to generate treatment chart information about the patient based on the diagnosis information received from the medical personnel account; and
a disease information reflection device configured to reflect the disease information to the treatment chart information by identifying the temporomandibular joint disease of the patient to identify the disease information corresponding to the identified temporomandibular joint disease among the plurality of disease information stored in the pre-stored database when the treatment chart information has been generated.

3. The system of claim 2, wherein the therapeutic agent suitability confirmation device includes:
a therapeutic agent attribute classification device configured to identify at least one digital therapeutic agent information matched to the identified disease information when a function of the disease information reflection device has been performed in order to classify the identified at least one digital therapeutic agent information according to an attribute; and
a therapeutic agent review request device configured to provide the medical personnel account with the at least one digital therapeutic agent information classified according to the attribute when the at least one digital therapeutic agent information is classified according to the attribute, in order to request the medical personnel account to review whether each of the at least one digital therapeutic agent information classified according to the attribute is suitable for the treatment of the temporomandibular joint disease.

4. The system of claim 3, wherein the attribute includes:
a substitution attribute which is used alone or in combination with an existing treatment for a specific disease to increase a therapeutic effect, as a reference for classification of the at least one digital therapeutic agent information; and
a complementary attribute that increases a therapeutic effect of an existing medication.

5. The system of claim 4, wherein the digital therapeutic agent management device includes:
a validity period confirmation device which, when an input indicating that each of the at least one digital therapeutic agent information classified according to the attribute is suitable for the treatment of the temporomandibular joint disease is received from the medical personnel account, confirms the validity period by identifying validity information matched to each of the at least one digital therapeutic agent information classified according to the attribute, based on the input;
a membership registration request device configured to provide the electronic device of the patient with the link for installing the digital therapeutic agent when the validity period has been confirmed in order to allow the patient to sign up for membership to use the digital therapeutic agent when the digital therapeutic agent has been installed through the link; and
a validity period allowance device configured to receive membership information created by the patient when signing up for the membership to use the digital therapeutic agent by performing a function of the membership registration request device in order to perform a validity period authentication process for the digital therapeutic agent through the received membership information, and to allow the patient to use the digital therapeutic agent during the confirmed validity period when the validity period authentication process has been completed.

6. The system of claim 5, wherein the prescription content providing device includes:
an intermediate information reception device configured to receive, from the electronic device, image information about a chin region captured through the electronic device and abnormality information created based on abnormality of the chin region, when the patient receives treatment of the temporomandibular joint disease through the digital therapeutic agent during the validity period; and
a survey information reception device configured to receive, from the electronic device, symptom survey information created based on a survey provided through the digital therapeutic agent, when the patient receives the treatment of the temporomandibular joint disease through the digital therapeutic agent during the validity period.

7. The system of claim 6, wherein the prescription content providing device further includes:
an intermediate treatment information transmission device configured to provide the medical personnel account with the received symptom survey information in a state in which the pain point has been identified, in order to allow the medical personnel account to remotely treat a progress of the temporomandibular joint disease corresponding to the pain point to transmit intermediate treatment information based on an intermediate treatment result to the electronic device.

8. The system of claim 6, wherein when the patient visits the hospital and receives treatment from a doctor who is a user of the medical personnel account, the prescription content providing device allows the medical personnel account to create a prescription for managing the temporomandibular joint disease of the patient in order to receive prescription content information based on the created prescription, identifies a prescription content corresponding to the prescription content information among a plurality of prescription contents stored in a pre-stored content database, and transmits a link for installing the identified prescription content to the electronic device.
